SEQUENCE LISTING (4 sequences)

SEQ ID NO: 1 - DNA, 860 bp, Homo sapiens
  CDS (1)..(738)
atgaaggtgcctcaatcaggaaccctgaagctgtcccacctacaggagggaacctacaccttccagctgaccgtgacggacactgccgggcagagaagctctgacaacgtgtcagtgacagtgcttcgcgcagcctactccacaggaggatgtttgcacacttgctcacgctaccacttcttctgtgacgatggctgctgcattgacatcacgctcgcctgcgatggagtgcagcagtgtcctgatgggtctgatgaagacttctgccagaatctgggcctggaccgcaagatggtaacccacacggcagctagtcctgccctgccaagaaccacagggccgagtgaagatgcagggggtgactccttggtggaaaagtctcagaaagccactgccccaaacaagccacctgcattatcaaacacagagaagaggaatcattccgccttttggggaccagagagtcaaatcattcctgtgatgccagatagtagttcctcagggaagaacagaaaagaggaaagttatatatttgagtcaaagggtgatggaggaggaggggaacacccagccccagaaacaggtgcagtgctacccctggcgctgggtttggctatcactgctctgctgcttctcatggttgcatgccgactacgactggtgaaacagaaactgaaaaaagctcgtcccattacatctgaggaatcggactacctcataaatgggatgtatctatagtaatgtaatttcaataccttggggcagggacatgttttgtttataatttatacatctattaagttctggatatttacagcttcttttgtttttaattgggccagaagattctgcaaatccca

SEQ ID NO: 2 - protein, 245 aa, Homo sapiens
MKVPQSGTLKLSHLQEGTYTFQLTVTDTAGQRSSDNVSVTVLRAAYSTGGCLHTCSRYHFFCDDGCCIDITLACDGVQQCPDGSDEDFCQNLGLDRKMVTHTAASPALPRTTGPSEDAGGDSLVEKSQKATAPNKPPALSNTEKRNHSAFWGPESQIIPVMPDSSSSGKNRKEESYIFESKGDGGGGEHPAPETGAVLPLALGLAITALLLLMVACRLRLVKQKLKKARPITSEESDYLINGMYL

SEQ ID NO: 3 - DNA, 1651 bp, Homo sapiens
  CDS (27)..(1529)
gctcgctgcccttggctctcgtcgccatggcctccgtcgcccaggagagcgcgggctcgcagcgccggctaccgccgcgtcacggggcgctgcgcgggctgctactgctctgcctgtggctgccaagcggccgtgcggccttgccgcccgcggcgccgctgtccgaactgcacgcgcagctgtcgggcgtggagcagctgctggaggagttccgccggcaactgcagcaggagcggcctcaggaggagctggagctggagctgcgtgcgggcggcggcccccaggaggactgcccgggcccgggcagcggcggctacagcgcaatgcctgacgccatcatccgcaccaaggactccctggcggcgggtgccagcttcctgcgggcgccggcggccgtgcggggctggcggcaatgcgtggcggcctgctgctccgagccgcgctgctccgtggccgtggtggagctgccccggcgccccgcgcccccggcagccgtgctcggctgctacctcttcaactgcacggcgcgcggccgcaacgtctgcaagttcgcgctgcacagcggctacagcagctacagcctcagccgcgcgccggacggcgccgccctggccaccgcgcgcgcctcgccccggcaggaaaaggatgcgcctccacttagcaaggctgggcaggatgtggttctgcatctgcccacagacggggtggttctagacggccgcgagagcacagatgaccacgccatcgtccagtatgagtgggcactgctgcagggggacccgtcagtggacatgaaggtgcctcaatcaggaaccctgaagctgtcccacctacaggagggaacctacaccttccagctgaccgtgacggacactgccgggcagagaagctctgacaacgtgtcagtgacagtgcttcgcgcagcctactccacaggaggatgtttgcacacttgctcacgctaccacttcttctgtgacgatggctgctgcattgacatcacgctcgcctgcgatggagtgcagcagtgtcctgatgggtctgatgaagacttctgccagaatctgggcctggaccgcaagatggtaacccacacggcagctagtcctgccctgccaagaaccacagggccgagtgaagatgcagggggtgactccttggtggaaaagtctcagaaagccactgccccaaacaagccacctgcattatcaaacacagagaagaggaatcattccgccttttggggaccagagagtcaaatcattcctgtgatgccagatagtagttcctcagggaagaacagaaaagaggaaagttatatatttgagtcaaagggtgatggaggaggaggggaacacccagccccagaaacaggtgcagtgctacccctggcgctgggtttggctatcactgctctgctgcttctcatggttgcatgccgactacgactggtgaaacagaaactgaaaaaagctcgtcccattacatctgaggaatcggactacctcataaatgggatgtatctatagtaatgtaatttcaataccttggggcagggacatgttttgtttataatttatacatctattaagttctggatatttacagcttcttttgtttttaattgggccagaagattctgcaaatccca

SEQ ID NO: 4 - protein, 500 aa, Homo sapiens
MASVAQESAGSQRRLPPRHGALRGLLLLCLWLPSGRAALPPAAPLSELHAQLSGVEQLLEEFRRQLQQERPQEELELELRAGGGPQEDCPGPGSGGYSAMPDAIIRTKDSLAAGASFLRAPAAVRGWRQCVAACCSEPRCSVAVVELPRRPAPPAAVLGCYLFNCTARGRNVCKFALHSGYSSYSLSRAPDGAALATARASPRQEKDAPPLSKAGQDVVLHLPTDGVVLDGRESTDDHAIVQYEWALLQGDPSVDMKVPQSGTLKLSHLQEGTYTFQLTVTDTAGQRSSDNVSVTVLRAAYSTGGCLHTCSRYHFFCDDGCCIDITLACDGVQQCPDGSDEDFCQNLGLDRKMVTHTAASPALPRTTGPSEDAGGDSLVEKSQKATAPNKPPALSNTEKRNHSAFWGPESQIIPVMPDSSSSGKNRKEESYIFESKGDGGGGEHPAPETGAVLPLALGLAITALLLLMVACRLRLVKQKLKKARPITSEESDYLINGMYL

(19) United States
(12) Patent Application Publication
(10) Pub. No.: US 2003/0208051 A1
(43) Pub. Date: Nov. 6, 2003

(54) Human hepatocyte growth factor activator inhibitor homologue

(76) Inventor: Eric Wen Su, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/203,875

(22) PCT Filed: Feb. 27, 2001
(86) PCT No.: PCT/US01/06301

(51) Int. Cl. C07K 14/475; C12P 21/02; C12N 5/06; C07H 21/04

(52) U.S. Cl. 530/399; 536/23.5; 435/69.1; 435/320.1; 435/325

(57) ABSTRACT

The present invention provides nucleic acid sequences encoding novel human hepatocyte growth factor activator inhibitors (HGF-AIh). These novel nucleic acids are useful for constructing the claimed DNA vectors and host cells of the invention and for preparing the claimed recombinant proteins and antibodies.

### FIELD OF INVENTION

The present invention relates to molecular biology as it applies to pharmaceutical research and development. The invention provides novel DNA sequences that encode new homologues of human hepatocyte growth factor activator inhibitor (HGF-AIh), host cells, transgenics, antibodies, compositions, and methods of making and using the foregoing.

### BACKGROUND OF THE INVENTION

Hepatocyte growth factor (HGF) is activated in response to tissue injury and is believed to promote healing in a variety of damaged tissues. HGF is secreted from producing cells as an inactive single chain precursor that associates with the extracellular matrix and is converted to a biologically active heterodimer by a liver-produced serine protease. Shimomura, et al., Eur J Biochem, 1;229(1):257-61,(1995). This serine protease, called HGF activator, circulates as an inactive zymogen in the blood and is converted to the active form by limited proteolysis. A Kunitz-type serine protease inhibitor (HAI-1) was isolated from the conditioned medium of a human stomach carcinoma cell line, MKN45, and is believed to participate in regulating the action of HGF via inhibitory effects on HGF activator. Shimomura, et al., J Biol Chem, 272(10):6370-6 (1997). A second Kunitz-type HGF activator inhibitor (HAI-2) was isolated from the MKN45 carcinoma cell line and was found to share certain properties with HAI-1. Molecular cloning revealed that HAI-2 is derived from a 252 amino acid precursor that contains two Kunitz-type domains and a hydrophobic COOH-terminal region. Kawaguchi et al., J Biol Chem, 272(44):27558-64 (1997).

Studies have shown that HAI-1 expression is lower in the white matter of Alzheimer brain tissue compared to normal brain white matter. Yamada et al., Exp Neurol, 153(1):60-4 (1998). Other studies have demonstrated that adenocarcinoma tissue expresses lower levels of HAI-1 than in their normal counterparts. Kataoke et al., Cancer Lett, 128(2):219-27 (1998). Thus HAI and related proteins are clearly indicated as having a great potential for treating diseases involving poorly regulated cellular proliferation.

More generally, all novel proteins are of interest. Proteins of course play an important nutritional role for animals. Extracellular proteins are critical players in the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Secreted proteins have various industrial applications, including pharmaceuticals, diagnostics, biosensors and bioreactors. Many protein drugs are available at present, such as insulin, growth hormone, thrombolytic agents, interferons, interleukins, erythropoietins, colony stimulating factors, and various other cytokines, all of which are secreted proteins. Membrane-bound protein receptors and fragments thereof, such as ENBREL® have also been proven as therapeutic and diagnostic agents.

Efforts are being undertaken by both industry and academia to identify new, native secreted proteins. Many efforts are focused on the screening of mammalian recombinant DNA libraries to identify the coding sequences for novel secreted proteins. Examples of screening methods and techniques are described in the literature. See, for example, Klein et al., Proc Natl Acad Sci, B:7108-7113 (1996) and U.S. Pat. No. 5,536,637. The results of such efforts are presented herein.

HGF-Aih shows homology to the C-terminal region of HAI-1 where one of the Kunitz-type serine protease domains is found. HGF-Aih also shows some homology to a 313 base pair expressed sequence tag (EST) clone, CI294, disclosed in WO 9845436-A2. Northern blot analysis established that HGF-Aih is expressed in a wide variety of normal tissues.

### BRIEF SUMMARY OF THE INVENTION

The present invention provides nucleic acid sequences encoding novel human hepatocyte growth factor activator inhibitors (HGF-AIh). These novel nucleic acids are useful for constructing the claimed DNA vectors and host cells of the invention and for preparing the claimed recombinant proteins and antibodies. In particular, a human EST cDNA clone is disclosed that contains an open reading frame encoding a 245 amino acid homologue of human hepatocyte growth factor activator inhibitor. Also, a full length human cDNA clone is disclosed that contains an open reading frame encoding a 500 amino acid homologue of human hepatocyte growth factor activator inhibitor.

### DETAILED DESCRIPTION OF THE INVENTION

The present inventors have identified a 2521 base pair cDNA clone, SEQ ID NO: 1, that contains a 735 base pair open reading encoding a novel 245 amino acid polypeptide, HGF-AIh, having sequence identity with HAI-1 and 2.

The present inventors have identified a 3264 base pair cDNA clone, SEQ ID NO: 3, that contains a 1500 base pair open reading encoding a novel 500 amino acid polypeptide (SEQ ID NO: 4), HGF-AIh, having sequence identity with HAI-1 and 2. The invention further provides a particular HGF-AIh polypeptide sequence, SEQ ID NO: 4.

The terms “HGF-AIh polypeptide”, and “HGF-AIh” when used herein encompass native sequence HGF-AIh polypeptide and polypeptide variants thereof (which are further defined herein). The HGF-AIh polypeptides may be isolated from a variety of sources, such as from human tissue types or from another source, or prepared by recombinant or synthetic methods.

A “native sequence HGF-AIh polypeptide” comprises a polypeptide having the same amino acid sequence as an HGF-AIh polypeptide, derived from nature. Such native sequence HGF-AIh polypeptide can be isolated from nature or can be produced by recombinant or synthetic means. The term “native sequence HGF-AIh polypeptide” specifically encompasses naturally-occurring truncated or secreted forms of an HGF-AIh polypeptide, (e.g., soluble forms containing for instance, an extracellular domain sequence), naturally-occurring variant forms (e.g., alternatively spliced forms) and naturally-occurring allelic variants of an HGF-AIh polypeptide.

In one embodiment of the invention, the native sequence HGF-AIh polypeptide is a full-length or mature native sequence HGF-AIh polypeptide comprising amino acids 1 through 245 of SEQ ID NO: 2. In another embodiment, a native HGF-AIh polypeptide sequence is a full-length or mature native HGF-AIh polypeptide comprising amino acids 1 through 500 of SEQ ID NO: 4. Also, while an HGF-AIh polypeptide can be shown to begin with a methionine residue designated as amino acid position 1, it is conceivable and possible that another methionine residue located either upstream or downstream from amino acid position 1 in SEQ ID NO: 2 or SEQ ID NO: 4 may be employed as the starting amino acid residue.

The term HGF-AIh refers to a specific native sequence HGF-AIh polypeptide depicted, respectively, in SEQ ID NO: 2 or in SEQ ID NO: 4.

“HGF-AIh variant” means an “active” HGF-AIh polypeptide as defined below having at least about 80% amino acid sequence identity with the HGF-AIh polypeptide respectively, having the deduced amino acid sequence of residues 1 to about 245 shown in SEQ ID NO: 2 or residues 1 or about 37 to about 500 shown in SEQ ID NO: 4, for a full-length or mature native sequence HGF-AIh polypeptide. Such HGF-AIh polypeptide variants include, for instance, HGF-AIh, wherein one or more amino acid residues are added, substituted or deleted, at the N- or C-terminus or within the sequence of SEQ ID NO: 2 or SEQ ID NO: 4. Ordinarily, an HGF-AIh polypeptide variant will have at least about 80% amino acid sequence identity, preferably at least about 81% amino acid sequence identity, more preferably at least about 82% amino acid sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 96% sequence identity, yet more preferably at least about 97% sequence identity, yet more preferably at least about 98% sequence identity, yet more preferably at least about 99% ammo acid sequence identity with the amino acid sequence of SEQ ID NO: 2 or of SEQ ID NO: 4 (with or without the signal peptide). The variants provided herein exclude native sequence HGF-AIh sequence as well the polypeptides and nucleic acids described herein with which the HGF-AIh polypeptides share 100% identity and/or which are already known in the art.

“Percent (%) amino acid sequence identity” with respect to the HGF-AIh amino acid sequences identified herein is defined as the percentage of amino acid residues in a candidate sequence that are identical with the amino acid residues in an HGF-AIh polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. For example, the % identity values used herein are generated using WU-BLAST-2 (Altschul et al.) Methods in Enzymology 266: 460-480 (1996). Most of the WU-BLAST-2 search parameters are set to the default values. Those not set to default values, i.e., the adjustable parameters, are set with the following values: overlap span=1. overlap fraction=0.125: word threshold (T)=11, and scoring matrix=BLOSUM 62. For purposes herein, a % amino acid sequence identity value is determined by divided (a) the number of matching identical ammo acid residues between the amino acid sequence of the HGF-AIh polypeptide of interest and the comparison amino acid sequence of interest (i.e., the sequence against which the HGF-AIh polypeptide of interest is being compared) as determined by WU-BLAST-2 by (b) the total number of amino acid residues of the HGF-AIh polypeptide of interest, respectively.

A “HGF-AIh variant polynucleotide” or HGF-AIh variant nucleic acid sequence” means an active HGF-AIh polypeptide-encoding nucleic acid molecule as defined below having at least about 65% nucleic acid sequence identity with the nucleotide acid sequence of nucleotides about 145 to about 879 of the HGF-AIh-encoding nucleotide sequence shown in SEQ ID NO: 1 or an active HGF-AIh polypeptide-encoding nucleic acid molecule having at least about 65% nucleic acid sequence identity with the nucleotide acid sequence of nucleotides about 147 or about 258 to about 1647 of the HGF-AIh-encoding nucleotide sequence shown in SEQ ID NO: 3. Ordinarily, an HGF-AIh polypeptide will have at least about 65% nucleic acid sequence identity, more preferably at least about 70% nucleic acid sequence identity, yet more preferably at least about 75% nucleic acid sequence identity, yet more preferably at least about 80% nucleic acid sequence identity, yet more preferably at least about 81% nucleic acid sequence identity, yet more preferably at least about 82% nucleic acid sequence identity, yet more preferably at least about 83% nucleic acid sequence identity, yet more preferably at least about 84% nucleic acid sequence identity, yet more preferably at least about 85% nucleic acid sequence identity, yet more preferably at least about 86% nucleic acid sequence identity, yet more preferably at least about 87% nucleic acid sequence identity, yet more preferably at least about 88% nucleic acid sequence identity, yet more preferably at least about 89% nucleic acid sequence identity, yet more preferably at least about 90% nucleic acid sequence identity, yet more preferably at least about 91% nucleic acid sequence identity, yet more preferably at least about 92% nucleic acid sequence identity, yet more preferably at least about 93% nucleic acid sequence identity, yet more preferably at least about 94% nucleic acid sequence identity, yet more preferably at least about 95% nucleic acid sequence identity, yet more preferably at least about 96% nucleic acid sequence identity, yet more preferably at least about 97% nucleic acid sequence identity, yet more preferably at least about 98% nucleic acid sequence identity, yet more preferably at least about 99% nucleic acid sequence identity with the nucleic acid sequence of nucleotides about 145 to about 789 of the HGF-AIh-encoding nucleotide sequence shown in SEQ ID NO: 1 or at least about 99% nucleic acid sequence identity with the nucleic acid sequence of nucleotides about 147 or about 258 to about 1647 of the sequence shown in SEQ ID NO: 3. Variants specifically exclude or do not encompass the native nucleotide sequence, as well as those prior art sequences that share 100% identity with the nucleotide sequences of the invention.

“Percent (%) nucleic acid sequence identity” with respect to the HGF-AIh sequences identified herein is defined as the percentage of nucleotides in a candidate sequence that are identical with the nucleotides in the HGF-AIh sequence after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN, Align-2, Megalign (DNASTAR), or BLAST (e.g., Blast, Blast-2) software. Those skilled in the art can determine appropriate parameters for measuring alignment, including any algorithms needed to achieve maximal alignment over the full length of the sequences being compared. For purposes herein, however, % nucleic acid identity values are generated using the WU-BLAST-2 (BlastN module) computer program (Altschul et al., Methods in Enzymology m: 460-480 (1996). Most of the WU-BLAST-2 search parameters are set to the default values. Those not set default values, i.e., the adjustable parameters, are set with the following values: overlap span=1, overlap fraction=0.125, word threshold (T)=11 and scoring matrix=BLOSUM62. For purposes herein, a % nucleic acid sequence identity value is determined by dividing (a) the number of matching identical nucleotides between the nucleic acid sequence of the PRO polypeptide-encoding nucleic acid molecule of interest and the comparison nucleic acid molecule of interest (i.e., the sequence against which the PRO polypeptide-encoding nucleic acid molecule of interest is being compared) as determined by WU-BLAST-2 by (b) the total number of nucleotides of the HGF-AIh polypeptide-encoding nucleic acid molecule of interest.

In other embodiments, the HGF-AIh variant polypeptides are nucleic acid molecules that encode an active HGF-AIh polypeptide and which are capable of hybridizing, preferably under stringent hybridization and wash conditions, to nucleotide sequences encoding a full-length HGF-AIh polypeptide shown in SEQ ID NO: 2 or a mature polypeptide of SEQ ID NO: 4. This scope of variant polynucleotides specifically excludes those sequences that are known as of the filing and/or priority dates of the present application. Furthermore, HGF-AIh variant polypeptides may also be those that are encoded by an HGF-AIh variant polynucleotides, respectively.

The term “positives”, in the context of sequence comparison performed as described above, includes residues in the sequences compared that are not identical but have similar properties (e.g., as a result of conservative substitutions). The % identity value of positives is determined by the fraction of residues scoring a positive value in the BLOSUM 62 matrix. This value is determined by dividing (a) the number of amino acid residues scoring a positive value in the BLOSUM62 matrix of WU-BLAST-2 between the HGF-AIh polypeptide amino acid sequence of interest and the comparison amino acid sequence (i.e., the amino acid sequence against which the HGF-AIh polypeptide sequence is being compared) as determined by WU-BLAST-2 by (b) the total number of amino acid residues of the HGF-AIh polypeptide of interest.

“Isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under nonreducing or reducing conditions using Coomassie blue or, preferably, silver stain. Isolated polypeptide includes polypeptide in situ within recombinant cells, since at least one component of the HGF-AIh polypeptide natural environment will not be present. Ordinarily, however, isolated polypeptide will be prepared by at least one purification step.

An “isolated” HGF-AIh polypeptide-encoding nucleic acid molecule is a nucleic acid molecule that is identified and separated from at least one contaminant nucleic acid molecule with which it is ordinarily associated in the natural source of the HGF-AIh polypeptide-encoding nucleic acid. An isolated HGF-AIh polypeptide-encoding nucleic acid molecule is other than in the form or setting in which it is found in nature. Isolated HGF-AIh polypeptide-encoding nucleic acid molecules therefore are distinguished from the HGF-AIh polypeptide-encoding nucleic acid molecule as it exists in natural cells. However, an isolated HGF-AIh polypeptide-encoding nucleic acid molecule includes HGF-AIh polypeptide encoding nucleic acid molecules contained in cells that ordinarily express HGF-AIh polypeptide polypeptide, where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes required higher temperatures for proper annealing, while short probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reactions more stringent, while lower temperatures less so. For additional details and explanation of stringency of hybridization reactions, see Ausubel et al., Current Protocols in Molecular Biology, Wiley Inter-science Publishers, (1995).

“Stringent conditions” or “high stringency conditions”, as defined herein, may be identified by those that” (1) employ low ionic strength and high temperature for washing, for example 0.015 M sodium chloride/0.001 5 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M 2% NaCl, 0.075 M sodium citrate), 50 mM sodium phosphate (pH 6.8), 0.1% sodium pyrophosphate, 5× Denhardt's solution, sonicated salmon sperm DNA (50 ug/ml), 0.1% SDS, and 10% dextmn sulfate at 42° C. with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” where used herein refers to a chimeric polypeptide comprising an HGF-AIh polypeptide, or domain sequence thereof, fused to a “tag polypeptide”. The tag polypeptide has enough residues to provide an epitope against which an antibody may be made, or which can be identified by some other agent, yet is short enough such that it does not interfere with the activity of the HGF-AIh polypeptide. The tag polypeptide preferably is also fairly unique so that the antibody does not substantially cross-react with other epitopes. Suitable tag polypeptides generally have at least six amino acid residues and usually between about 8 to about 50 amino acid residues (preferably, between about 10 to about 20 residues).

As used herein, the term “immunoadhesin” designates antibody-like molecules that combine the binding specificity of a heterologous protein (an “adhesion”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3 or IgG-4 subtypes, IgA (including IgG-1 and IgA-2), IgE, IgD or IgM.

The term “antibody” is used in the broadest sense and specifically covers single anti-HGF-AIh polypeptide monoclonal antibodies (including agonist, antagonist, and neutralizing antibodies), anti-HGF-AIh, respectively, antibody compositions with polyepitopic specificity, single-chain anti-HGF-AIh antibodies, and fragments of anti-HGF-AIh antibodies. The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Active” or “activity” for the purposes herein refers to form(s) of HGF-AIh which retain the biologic and/or immunologic activities of native or naturally-occurring HGF-AIh, respectively, polypeptide. Elaborating further, “biological” activity refers to a biological function (either inhibitory or stimulatory) caused by a native or naturally-occurring HGF-AIh other than the ability to induce the production of an antibody against an antigenic epitope possessed by a native or naturally-occurring HGF-AIh and an “immunological” activity refers only to the ability to induce the production of an antibody against an antigenic epitope possessed by a native or naturally-occurring HGF-AIh. A preferred biological activity includes the ability to inhibit growth factor activators resulting in homeostatic regulation of cellular proliferation.

The term “antagonist” is used in the broadest sense, and includes any molecule that partially or fully blocks, inhibits, or neutralizes a biological activity of a native HGF-AIh polypeptide disclosed herein. In a similar manner, the term “agonist” is used in the broadest sense and includes any molecule that mimics a biological activity of a native HGF-AIh polypeptide disclosed herein. Suitable agonist or antagonist molecules specifically include agonist or antagonist antibodies or antibody fragments, fragments or amino acid sequence variants of native HGF-AIh polypeptides, peptides, small organic molecules, etc. Methods for identifying agonists or antagonists of an HGF-AIh polypeptide may comprise contacting an HGF-AIh polypeptide with a candidate agonist or antagonist molecule and measuring a detectable change in one or more biological activities normally associated with the HGF-AIh polypeptide.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. The term “antibody” is used in the broadest sense and specifically covers, without limitation, intact monoclonal antibodies, polyclonal antibodies. multispecific antibodies (e.g. bispecific antibodies) formed from at least two intact antibodies, and antibody fragments so long as they exhibit the desired biological activity. The term antibody includes chimeric, humanized and fully human antibodies.

The terms “treating”, “treatment” and “therapy” as used herein refer to curative therapy, prophylactic therapy, and preventative therapy. An example of “preventative therapy” is the prevention or lessened targeted pathological condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

The term “mammal” as used herein refers to any mammal classified as a mammal, including humans, domestic and farm animals, and zoo, sports or pet animals, such as cattle (e.g. cows), horses, dogs, sheep, pigs, rabbits, goats. cats, etc. In a preferred embodiment of the invention, the mammal is a human.

Administration “in combination with” one or more further therapeutic agents includes simultaneous (concurrent) and consecutive administration in any order.

A “therapeutically-effective amount” is the minimal amount of active agent (e.g., an HGF-AIh polypeptide, antagonist or agonist thereof) which is necessary to impart therapeutic benefit to a mammal. For example a “therapeutically-effective amount” to a mammal suffering or prone to suffering or to prevent it from suffering from a proliferative disorder is such an amount which induces, ameliorates or otherwise causes an improvement in the pathological symptoms, disease progression, physiological conditions associated with or resistance to succumbing to a disorder principally characterized by poorly or unregulated cellular proliferation

“Carriers” as used herein include pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine. glutamine, asparagine, arginine or lysine; monosaccharides, disaccharides, and other carbohydrates including glucose. mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)1 and Fv fragments; diabodies; linear antibodies (Zapata et al., Protein Engin. S(10): 1057-1062 [19951); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment, which contains a complete antigen-recognition and binding site. This region consists of a dimer of one heavy- and one light chain variable domain in tight, non-covalent association. It is in this configuration that the three complimentarity-determining regions (CDRs) of each variable domain interact to define an antigen-binding site on the surface of the VH-VL dimer. Collectively, the six CDRs confer antigen-binding specificity to the antibody. However, even a single variable domain (or half of an Fv comprising only three CDR specific for an antigen) has the ability to recognize and bind antigen, although at a lower affinity than the entire binding site.

The Fab fragment also contains the constant domain of the light chain and the ‘first constant domain (CHI) of the heavy chain. Fab fragments differ from Fv fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)_{z} antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domain, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-3 15 (1994).

The term “diabodies” refers to small antibody fragments with two antigen-binding sites, which fragments comprise a heavy-chain variable domain (VH) connected to a lightchain variable domain (VL) in the same polypeptide chain (VH-VL). By using a linker that is too short to allow pairing between the two domains on the same chain, the domains are forced to pair with the complementary domains of another chain and create two antigen binding sites. Diabodies are described more fully in, for example, EP 404.097, WO 93/1 1161; and Hollinger et al., Proc. NatI. Acad. Sci. USA 90: 6444-6448 (1993).

An “isolated” antibody is one, which has been identified and separated and/or recovered from a component of its natural environment. Contaminant components of its natural environment are materials, which would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method. and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or preferably, silver stain. Isolated antibody includes the antibody in situ within recombinant cells since at least one component of the antibody's natural environment will not be present. Ordinarily, however. isolated antibody will be prepared by at least one purification step.

The word “label” when used herein refers to a detectable compound or composition which is conjugated directly or indirectly to the antibody so as to generate a “labeled” antibody. The label may be detectable by itself (e.g., radioisotope labels or fluorescent labels) or, in the case of an enzymatic label. may catalyze chemical alternation of a substrate compound or composition, which is detectable.

“Solid phase” is meant to be a non-aqueous matrix to which the antibody of the present invention can adhere. Examples of solid phases encompassed herein include those formed partially or entirely of glass (e.g., controlled pore glass). polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromotagraphy column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as an HGF-AIh polypeptide or antibody thereto) to a mammal. The components of the liposome are commonly arranged in a bilayer formation, similar to the lipid arrangement of biological membranes.

A “small organic molecule” is defined herein to have a molecule weight below about 500 Daltons.

The term “modulate” means to affect (e.g., either upregulate, downregulate or otherwise control) the level of a signaling pathway. Cellular processes under the control of signal transduction include, but are not limited to. transcription of specific genes, normal cellular functions, such as metabolism, proliferation, differentiation. adhesion, apoptosis and survival, as well as abnormal processes, such as transformation, blocking of differentiation and metastasis.

In one aspect, an isolated nucleic acid comprises DNA encoding the HGF-AIh polypeptide having amino acid residues from 145 through 879 of SEQ ID NO: 1 or from about 147 to about 258 through 1647 of SEQ ID NO: 3 or is complementary to either of such encoding nucleic acid sequences, and remains bound in a stable manner to it under at least moderate, and optionally, under high stringency conditions.

In another embodiment, the isolated nucleic acid comprises DNA having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity. yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity. yet more preferably at least about 97% sequence identity, yet more preferably at least about 98% sequence identity, yet more preferably at least about 99% sequence identity to (a) a DNA molecule (encoding a polypeptide comprising a sequence of amino acid residues) from 145 to 879, inclusive, of SEQ ID NO: 1 or a DNA molecule (encoding a polypeptide comprising a sequence of amino acid residues) from about 147 or about 258 through 1647, inclusive, of SEQ ID NO: 3 or (b) the complement of the DNA molecule of (a). Alternatively, an isolated nucleic acid comprises DNA encoding an HGF-AIh polypeptide having the sequence of amino acid residues from about 1 to about 245, inclusive of SEQ ID NO: 2 or an isolated nucleic acid comprises DNA encoding a polypeptide from about 1 or about 37 to about 500, inclusive of SEQ ID NO: 4.

In a further aspect, the invention concerns an isolated nucleic acid molecule produced by hybridizing a test DNA molecule under stringent conditions with: (a) a DNA molecule encoding (i) an HGF-AIh polypeptide having the sequence of amino acid residues from about 145 to about 879, inclusive, SEQ ID NO: 1, or (b) the complement of the DNA molecule of (a), and if the DNA molecule has at least about an 80% sequence identity, preferably at least about an 81% sequence identity, more preferably at least about a 82% sequence identity, yet more preferably at least about a 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity. yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% sequence identity to (a) or (b), and isolating the test DNA molecule.

In yet a further aspect, the invention concerns an isolated nucleic acid molecule comprising: (a) DNA encoding a polypeptide scoring at least about 80% positives, preferably at least about 81% positives, more preferably at least about 82% positives, yet more preferably at least about 83% positives, yet more preferably at least about 84% positives, yet more preferably at least about 85% positives, yet more preferably at least about 86% positives, yet more preferably at least about 87% positives, yet more preferably at least about 88% positives, yet more preferably at least about 89% positives, yet more preferably at least about 90% positives, yet more preferably at least about 91% positives, yet more preferably at least about 92% positives, yet more preferably at least about 93% positives, yet more preferably at least about 94% positives, yet more preferably at least about 95% positives, yet more preferably at least about 96% positives, yet more preferably at least about 97% positives, yet more preferably at least about 98% positives, yet more preferably at least about 99% positives, when compared with the amino acid sequence of residues about (i) 1 to about 245 inclusive, SEQ ID NO: 2, or (ii) 1 or about 37 to about 500 inclusive, of SEQ ID NO: 4, or (b) the complement of the DNA of (a). In a specific aspect, the invention provides an isolated nucleic acid molecule comprising DNA encoding an HGF-AIh polypeptide without an N-terminal signal sequence and in another embodiment the invention provides an isolated nucleic acid molecule comprising DNA encoding an HGF-AIh polypeptide with an N-terminal signal sequence of about 37 amino acid residues and/or an initiating methionine, or is complementary to such encoding nucleic acid molecule. It is also recognized that, in some cases, cleavage of the signal sequence form a secreted polypeptide is not entirely uniform, resulting in more than one secreted species. These polypeptides, and the polynucleotides encoding them, are contemplated by the present invention.

Another embodiment is directed to fragments of an HGF-AIh-encoding sequence that may find use as, for example, hybridization probes or for encoding fragments of an HGF-AIh polypeptide that may optionally encode a polypeptide comprising a binding site for an anti-HGF-AIh antibody. Such nucleic acids fragments are usually at least about 20 nucleotides in length, preferably at least about 30 nucleotides in length, more preferable at least about 40 nucleotides in length, yet more preferably at least about 50 nucleotides in length, yet more preferably at least about 60 nucleotides in length, yet more preferably at least about 70 nucleotides in length, yet more preferably at least about 50 nucleotides in length, yet more preferably at least about 90 nucleotides in length, yet more preferably at least about 100 nucleotides in length, yet more preferably at least about 110 nucleotides in length, yet more preferably at least about 120 nucleotides in length, yet more preferably at least about 130 nucleotides in length, yet more preferably at least about 140 nucleotides in length, yet more preferably at least about 150 nucleotides in length, yet more preferably at least about 160 nucleotides in length, yet more preferably at least about 170 nucleotides in length, yet more preferably at least about 180 nucleotides in length, yet more preferably at least about 190 nucleotides in length, yet more preferably at least about 200 nucleotides in length, yet more preferably at least about 250 nucleotides in length, yet more preferably at least about 300 nucleotides in length, yet more preferably at least about 350 nucleotides in length, yet more preferably at least about 400 nucleotides in length, yet more preferably at least about 450 nucleotides in length, yet more preferably at least about 500 nucleotides in length, yet more preferably at least about 600 nucleotides in length, yet more preferably at least about 700 nucleotides in length, yet more preferably at least about 800 nucleotides in length, yet more preferably at least about 900 nucleotides in length, yet more preferably at least about 100 nucleotides in length, wherein in this context “about” means the referenced nucleotide sequence length plus or minus 10% of that referenced length. In a preferred embodiment, the nucleotide sequence fragment is derived from any coding region of the nucleotide sequence shown in SEQ ID NO: 1 or SEQ ID NO: 3. In a more preferred embodiment, the nucleotide sequence fragment is derived from nucleotides about 145 through about 879, inclusive of SEQ ID NO: 1 or from about about 147 or about 258 through 1647, inclusive of SEQ ID NO: 3.

In another embodiment, the invention provides a vector comprising DNA encoding an HGF-AIh. The vector may comprise any of the isolated nucleic acid molecules described herein.

In another embodiment, the invention provides a host cell comprising the above vector. By way of example, the host cells may be CHO cells, E. coli, or yeast. A process for producing HGF-AIh polypeptides is further provided and comprises culturing host cells under conditions suitable for expressing HGF-AIh, respectively, and recovering HGF-AIh, respectively, from the cell culture.

In another embodiment, the invention provides isolated HGF-AIh polypeptides encoded by any of the isolated nucleic acid sequences described herein.

In another aspect, the invention concerns an isolated HGF-AIh polypeptide, comprising an amino acid sequence having at least about 80% sequence identity, preferably at least about 81% sequence identity, more preferably about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% sequence identity to the sequence of amino acid residues about (a) 1 to about 245, inclusive, of SEQ ID NO: 2 or (b) 1 or about 37 to about 500, inclusive, of SEQ ID NO: 4.

In a further aspect, the invention concerns an isolated HGF-AIh polypeptide comprising an amino acid sequence scoring at least about 80% positives, preferably at least about 81% positives, more preferably at least about 82% positives, yet more preferably at least about 83% positive, yet more preferably at least about 84% positives. yet more preferably at least about 85% positives, yet more preferably at least about 86% positives, yet more preferably at least about 87% positives, yet more preferably at least about 88% positives, yet more preferably at least about 89% positives, yet more preferably at least about 90% positives, yet more preferably at least about 91% positives, yet more preferably at least about 92% positives, yet more preferably at least about 93% positives, yet more preferably at least about 94% positives, yet more preferably at least about 95% positives, yet more preferably at least about 96% positives, yet more preferably at least about 97% positives, yet more preferably at least about 98% positives, yet more preferably at least about 99% positives, when compared with the amino acid sequence of (a) residues from about 1 to about 245, inclusive, of SEQ ID NO: 2, or (b) residues from about 1 or about 37 to about 500, inclusive, of SEQ ID NO: 4.

In a specific aspect, the invention provides an isolated HGF-AIh polypeptide without an N-terminal signal sequence and/or initiating methionine and is encoded by a nucleotide sequence (SEQ ID NO: 1) that encodes such an amino acid sequence as previously discussed. In another aspect, the invention provides an isolated HGF-AIh polypeptide with an N-terminal signal sequence and/or initiating methionine of about 37 residues and is encoded by a nucleotide sequence (SEQ ID NO: 3) that encodes such an amino acid sequence as previously discussed. Processes for producing the same are also herein described, wherein those processes comprise culturing a host cell comprising a vector which comprises the appropriate encoding nucleic acid molecule under conditions suitable for expression of HFG-AIh polypeptide and recovering the HGF-AIh polypeptide, respectively, from the cell culture.

In yet another aspect, the invention concerns an isolated HFG-Aih polypeptide comprising the sequence of amino acid residues from about 1 to about 245 inclusive, of SEQ ID NO: 2, and an isolated HFG-Aih polypeptide comprising the sequence of amino acid residues from about 1 or about 37 to about 500 inclusive, of SEQ ID NO: 4, or a fragment thereof (of either SEQ ID NO: 2 or SEQ ID NO: 4) which is biologically active or sufficient to provide a binding site for an anti-HFG-AIh antibody, wherein the identification of HFG-AIh polypeptide or fragments thereof that possess biological activity or provide a binding site for an anti-HFG-AIh antibody may be accomplished in a routine manner using techniques which are well known in the art.

In another embodiment, the invention provides chimeric molecules comprising an HFG-AIh polypeptide fused to a heterologous polypeptide or amino acid sequence. An example of such a chimeric molecule comprises an HFG-AIh polypeptide, respectively, fused to an epitope tag sequence or a Fc region of an immunoglobulin.

In another embodiment, the invention provides an antibody that specifically binds to an HFG-AIh polypeptide or fragment thereof. Optionally, the antibody is a monoclonal antibody, chimeric antibody, humanized antibody, human antibody, or an antibody fragment or a single chain antibody.

In yet another embodiment, the invention concerns agonists and antagonists of a native HFG-AIh polypeptide. In a particular aspect, the agonist or antagonist is an anti-HFG-AIh antibody, or a small organic molecule.

In yet another embodiment, the invention concerns a method of identifying agonists or antagonists of a native HFG-AIh polypeptide, by contacting the native HFG-AIh polypeptide with a candidate molecule and monitoring a biological activity mediated by said polypeptide.

In still a further embodiment, the invention concerns a composition comprising an HFG-AIh polypeptide, or an agonist or antagonist as hereinabove defined, in combination with a suitable carrier. Preferably, the carrier is pharmaceutically acceptable.

In still a further embodiment, the invention concerns the use of an HFG-AIh polypeptide, or an agonist or antagonist thereof as hereinbefore described, or an anti-HFG-AIh antibody, for the preparation of a medicament useful in the treatment of a condition which is responsive to the HFG-AIh polypeptide or an agonist or antagonist thereof (e.g., anti-HFG-AIh antibody). In a particular aspect, the invention concerns the use of an HFG-AIh polypeptide, or an agonist or antagonist thereof in a method for treating a disorder based on poorly regulated cellular proliferation.

In still a further embodiment, the invention relates to a method of treating a cancer or Alzheimer's disease by administrating a therapeutically effective amount of an HFG-AIh polypeptide, agonist, or antagonist thereof to a mammal suffering from said disorder.

In still a further embodiment, the invention relates to HFG-AIh antagonists and/or agonist molecules. In one aspect, the invention provides a method of screening compounds that mimic HFG-AIh (agonists) or diminish the effect of the HFG-AIh (antagonists).

In still a further embodiment, the invention relates to a therapeutic composition comprising a therapeutically effective amount of HFG-AIh, antagonist or agonist thereof in combination with a pharmaceutically-acceptable carrier.

In still a further embodiment, the invention relates to an article of manufacture comprising a container, label and therapeutically effective amount of HFG-AIh, antagonist or agonist thereof in combination with a pharmaceutically-acceptable carrier.

In addition to the full-length native sequence HFG-AIh polypeptides described herein, it is contemplated that HGF-AIh variants can be prepared. HGF-AIh variants can be prepared by introducing appropriate nucleotide changes into the HGF-AIh-encoding DNA, or by synthesis of the desired HGF-AIh polypeptide. Those skilled in the art will appreciate that amino acid changes may alter post-translational processing of the HGF-AIh polypeptide, such as changing the number or position of glycosylation sites or altering the membrane anchoring characteristics.

Variations in the native full-length sequence of HGF-AIh or in various domains of the HGF-AIh polypeptide described herein can be made, for example, using any of the techniques and guidelines for conservative and non-conservative mutations set forth, for instance, in U.S. Pat. No. 5,364,934. Variations include substitutions, deletions or insertions of one or more codons encoding the HGF-AIh polypeptide that results in a change in the amino acid sequence of the HGF-AIh polypeptide as compared with the native sequence HGF-AIh. Optionally the variation is by substitution of at least one amino acid with any other amino acid in one or more of the domains of the HGF-AIh polypeptide. Guidance in determining which amino acid residue may be inserted. substituted or deleted without adversely affecting the desired activity may be found by comparing the sequence of the HGF-AIh polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions, or substitutions of amino acids in the sequence and testing the resulting variants for activity (such as in any of the in virus assays described in the Examples below) for activity exhibited by the full-length or mature native sequence.

HGF-AIh polypeptide fragments are provided herein. Such fragments may be truncated at the N-terminus or C-terminus, or may lack internal residues, for example, when compared with a full length or native protein. Certain fragments lack amino acid residues that are not essential for a desired biological activity of the HGF-AIh polypeptide.

HGF-AIh fragments may be prepared by any of a number of conventional techniques. Desired peptide fragments may be chemically synthesized. An alternative approach involves generating HGF-AIh fragments by enzymatic digestion, e.g., by treating the protein with an enzyme known to cleave proteins at sites defined by particular amino aid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, HGF-AIh polypeptide fragments share at least one biological and/or immunological activity with the native HGF-AIh polypeptide shown in SEQ ID NO: 2 or SEQ ID NO: 4.

In particular embodiments, conservative substitutions of interest are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1
Conservative Substitutions

| Original Residue | Example substitutions | Preferred Substitutions |
|---|---|---|
| Ala (A) | val, leu, ile | val |
| Mg (R) | lys, gln, asn | lys |
| Asn (N) | | gln |
| Asp (D) | glu | glu |
| Cys (C) | ser | ser |
| Gin (Q) | asn | asn |
| Glu (E) | asp | asp |
| Gly (G) | pro, ala | ala |
| His (H) | asn, gln, lys, arg | arg |
| Be (I) | leu, val, met, ala, phe, | leu |
| | norleucine | |
| Leu (L) | norleucine, ile, val, met, al | ile |
| | phe | |
| Lys (K) | arg, gln, asn, | arg |
| Met (M) | leu, phe, ile | leu |
| Phe (F) | leu, val, ile, ala, tyr | leu |
| Pro (P) | ala | ala |
| Ser (S) | dir | thr |
| Thr (T) | ser | ser |
| Trp (W) | tyr,phe | tyr |
| Tyr (Y) | trp,phe,thr,ser | phe |
| Val (V) | ile, leu, met, phe, ala | leu |
| | norleucine | |

Substantial modifications in function or immunological identity of the HGF-AIh polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: lys, ser, thr;

(2) neutral hydrophilic: cys, ser. thr;

(3) acidic: asp. glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites, or more preferably. into the remaining (non-conserved) sites.

The variations can be made using methods known in the art such as oligonucleotide-mediated (site-directed) mutagenesis. alanine scanning, and PCR mutagenesis. Site-directed mutagenesis [Carter et al., Nucl. Acid Res. 13:433 1 (1986); Zoller et al., Nucl. Acid Res. 6487 (1987)], cassette mutagenesis [Wells et al., Gene, 34:3 15 (1985)], restriction selection mutagenesis [Wells et al., Philos. Trans. 317:415 (1986)] or other known techniques can be performed on the cloned DNA to produce the HGF-AIh-encoding variant DNA.

Scanning amino acid analysis can also be employed to identify one or more amino acids along a contiguous sequence. Among the preferred scanning amino acids are relatively small neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions [Creighton. The Proteins, (W.H. Freeman & Co., N.Y.). If alanine substitution does not yield adequate amounts of variant, an isoteric amino acid can be used.

Covalent modifications of HGF-AIh polypeptides are included within the scope of this invention. One type of covalent modification includes reacting targeted amino acid residues of an HGF-AIh polypeptide with an organic derivatizing agent that is capable of reacting with selected side chains or the N- or C-terminal residues of an HGF-AIh polypeptide. Derivatization with bifunctional agents is useful, for instance, for crosslinking HGF-AIh to a water-insoluble support matrix or surface for use in the method for purifying anti-HGF-AIh antibodies, and vice-versa. Commonly used crosslinking agents include. e.g., 1,1-bis(diazo-acetyl)-2-phenylethane, glutaraldehyde, N-hydroxy-succinimide esters, for example, esters with 4-azidosalicylic acid, homobifunctional imidoesters. including disuccinimidyl esters such as 3,3′-dithiobis-(succinimidylproprionate). bifunctional maleimides such as bis-N-maleimido1,8-octane and agents such as methyl-3-(p-azidophenyI)-dithiolproprioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the a-amino groups of lysine, arginine, and histidine side chains [T. E. Creighton. Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the HGF-AIh polypeptide included within the scope of this invention comprises altering the native glycosylation pattern of the polypeptide. “Altering the native glycosylation pattern” is intended for purposes herein to mean deleting one or more carbohydrate moieties found in native sequence of HGF-AIh polypeptide, and/or adding one or more glycosylation sites that are not present in the native sequence HGF-AIh polypeptide. Additionally, the phrase includes qualitative changes in the glycosylation of the native proteins involving a change in the nature and proportions of the various carbohydrate moieties present.

Addition of glycosylation sites to HGF-AIh polypeptides may be accomplished by altering the amino acid sequence thereof. The alteration may be made, for example, by the addition of, or substitution by, one or more serine or threonine residues to the native sequence HGF-AIh polypeptide (for O-linked glycosylation sites). The HGF-AIh amino acid sequence may optionally be altered through changes at the DNA level, particularly by mutating the DNA encoding the HGF-AIh polypeptide at preselected bases such that codons are generated that will translate into the desired amino acids.

Another means of increasing the number of carbohydrate moieties on the HGF-AIh polypeptide is by chemical or enzymatic coupling of glycosides to the polypeptide. Such methods are described in the art, e.g., in WO 87105330, and in Aplin and Wriston, CRC Crit. Rev. Biochem., pp. 259-306 (1981).

Removal of carbohydrate moieties present on the HGF-AIh polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem.; 131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of HGF-AIh comprises linking the HGF-AIh polypeptide, respectively, to one of a variety of nonproteinaceous co-polymers, e.g., polyethylene glycol, polypropylene glycol, or polyoxyalkylenes, in the manner set forth, for example, in U.S. Pat. Nos. 4,640,835; 4,496,689: 4,301,144; 4.670,417; 4.791,192 or 4,179,337.

HGF-AIh polypeptides of the present invention may also be modified in a way to form chimeric molecules comprising an HGF-AIh polypeptide fused to another heterologous polypeptide or amino acid sequence. In one embodiment, such a chimeric molecule comprises a fusion of an HGF-AIh polypeptide with a tag polypeptide which provides an epitope to which an anti-tag antibody can selectively bind. The epitope tag is generally placed at the amino- or carboxyl-terminus of the HGF-AIh polypeptide. The presence of such epitope-tagged forms of an HGF-AIh polypeptide can be detected using an antibody against the tag polypeptide. Also, provision of the epitope tag enables the HGF-AIh polypeptide to be readily purified by affinity purification using an anti-tag antibody or another type of affinity matrix that binds to the epitope tag.

Various tag polypeptides and their respective antibodies are well known in the art. Examples include poly-histidine (poly-his) or poly-histidine-glycine (poly-his-gly) tags; the flu HA tag polypeptide and its antibody; the c-myc tag and the 8F9, 3C7, 6E10, G4, B7 and 9E10 antibodies thereto [Evan et al., Mol Cell Biol, 5:3610-3616 (1985)]; and the Herpes Simplex virus glycoprotein D (gD) tag and its antibody. Other tag polypeptides include the Flag-peptide [Hopp et al., BioTechnology, 6:1204-1210 (1988)]; the KT3 epitope peptide [Martin et al., Science, m:192-194 (1992)]; an cr-tubulin epitope peptide [Skinner et al., J. Biol. Chem., 266: 15 163-1 5 166 (1991)]; and the T7 gene 10 protein peptide tag [Lutz-Freyermuth et al., PNAS, USA, u:6393-6397 (1990)].

In an alternative embodiment, the chimeric molecule may comprise a fusion of an HGF-AIh polypeptide with an immunoglobulin or a particular region of an immunoglobulin. For a bivalent form of the chimeric molecule, such a fusion could be to the Fc region of an IgG molecule. The Ig fusions preferably include the substitution of a soluble transmembrane domain deleted or inactivated form of a PRO 1031 or PRO 1122 polypeptide in place of at least one variable region within an Ig molecule. In a particularly preferred embodiment. the immunoglobulin fusion includes the hinge, CH2 and CH3. or the hinge, CH1, CH2 and CH3 regions of an IgG1 molecule. For the production of immunoglobulin fusions see also U.S. Pat. No. 5,428,130, issued Jun. 27, 1995.

In yet a further embodiment, the HGF-AIh polypeptides of the present invention may also be modified in a way to form a chimeric molecule comprising an HGF-AIh polypeptide fused to a leucine zipper. Various leucine zipper polypeptides have been described in the art. See, e.g., Landschulz et al., Science 240:1759 (1988); WO 94/10308; Maniatis et al., Nature 341:24 (1989). It is believed that use of a leucine zipper fused to an HGF-Aih or PRO1I22 polypeptide may be desirable to assist in dimerizing or trimerizing soluble HGF-AIh polypeptide in solution. Those skilled in the art will appreciate that the leucine zipper may be fused at either the N- or C-terminal end of the HGF-AIh molecule.

The description below relates primarily to production of HGF-AIh by culturing cells transformed or transfected with a vector containing HGF-AIh polypeptide encoding nucleic acid. It is, of course. contemplated that alternative methods, which are well known in the art, may be employed to prepare HGF-AIh polypeptides. For instance, the HGF-AIh sequence, or portions thereof, may be produced by direct peptide synthesis using solid-phase techniques [see, e.g., Merrifield, J. Am. Chem. Soc., :2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of HGF-AIh polypeptides may be chemically synthesized separately and combined using chemical or enzymatic methods to produce a full-length HGF-AIh polypeptide.

DNA encoding an HGF-AIh polypeptide may be obtained from a cDNA library prepared from tissue believed to possess the HGF-AIh mRNA and to express it at a detectable level. Accordingly, human HGF-AIh-encoding DNA can be conveniently obtained from a cDNA library prepared from human tissue, such as described in the Examples. The HGF-AIh-encoding gene may also be obtained from a genomic library or by known synthetic procedures (e.g., automated synthetic procedures, oligonucleotide synthesis).

Libraries can be screened with probes (such as antibodies to an HGF-AIh polypeptide or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding HGF-AIh is to use PCR methodology.

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art and include the use of radiolabels like ³²P-labeled ATP, biotinylation or enzyme labeling.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein (e.g., through sequence alignment using computer software programs such as ALIGN, DNAstar. BLAST, BLAST-2, INHERIT and ALIGN-2 which employ various algorithms to measure homology).

Nucleic acid having protein coding sequence may be obtained by screening selected cDNA or genomic libraries using the deduced amino acid sequence disclosed herein for the first time, and, if necessary, using conventional primer extension procedures as described in Sambrook et al. supra, to detect precursors and processing intermediates of mRNA that may not have been reverse-transcribed into cDNA.

Host cells are transfected or transformed with expression or cloning vectors described herein for HGF-AIh polypeptide production and cultured in conventional nutrient media modified as appropriate for inducing promoters. selecting transformants, or amplifying the genes encoding the desired sequences. The culture conditions. such as media, temperature. pH and the like, can be selected by the skilled artisan without undue experimentation. In general, principles. protocols, and practical techniques for maximizing the productivity of cell cultures can be found in Mammalian Cell Biotechnology: A Practical Approach, M. Butler, ed. (IRL Press, 1991).

Methods of transfection are known to the ordinarily skilled artisan, for example, CaPO₄ and electroporation. Depending on the host cell used. transformation is performed using standard techniques appropriate to such cells. The calcium treatment employing calcium chloride or electroporation is generally used for prokaryotes or other cells that contain substantial cell-wall barriers. Infection with Agrobacterium tumefaciens is used for transformation of certain plant cells, as described by Shaw et al., Gene, 23:3 1.5 (1983) and WO 89/05859. General aspects of mammalian cell host system transformations have been described in U.S. Pat. No. 4,399,216. Transformations into yeast are typically carried out according to the method of Van Solingen et al., J Bact., 130:946 (1977). However, other methods for introducing DNA into cells. such as by nuclear microinjection, electroporation, bacterial protoplast fusion with intact cells, or polycations, e.g., polybrene, polyomithine may also be used.

Suitable host cells for cloning or expressing the nucleic acid in the vectors herein include prokaryote, yeast, or higher eukaryote cells. Suitable prokaryotes include but are not limited to eubacteria, such as Gram-negative or Gram-positive organisms, for example, Enterobacteriacea such as E. coli. Various E. coli strains are publicly available, such as E. coli K12 strain MM294 (ATCC 3 1.446); E. coli X1 776 (ATCC 3 1.537); E. coli strain W3 110 (ATCC 27.325) and K5 772 (ATCC 53.635). Other suitable prokaryotic host cells include Enterobacteriaceae such as Escherichia, e.g. E. coli, Enterobacter, Erwinia, Klebisella, Proteus, Salmonella, e.g., Salmonella typhimurium. Serratia, e.g., Serratia marcescans, and Shigeila, as well as Bacilli such as B. subtilis and B. lichentformis (e.g., B. licheniformis 4 1 P disclosed in DD266,7 10, published Apr. 12, 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3 110 may be modified to effect a genetic mutation in the genes encoding proteins endogenous to the host, with examples of such hosts including E. coli W3110 strain 1A2. which has the complete genotype ronA; E. coli W3 110 strain 9E4. which has the complete genotype ton4 ptr3; E. coli W3110 strain 27C7 (ATCC 55,244), which has the complete genotype tonA, ptr3 phoA E15 (argF-lac) 169 degp ompT/can′; E. coli W3110 strain 40B4, which is strain 37D6 with a non-kanamycin resistant degP deletion mutation; and an E. coli strain having mutant periplasmic protease disclosed in U.S. Pat. No. 4,946,783 issued Aug. 7, 1990. Alternatively, in vivo methods of cloning, e.g., PCR or other nucleic acid polymerase reactions, are suitable.

In addition to prokaryotes. eukaryotic microbes such as filamentous fungi or yeast are suitable cloning or expression hosts for HGF-AIh-vectors. Saccharomyces cerevisiae is a commonly used lower eukaryotic host microorganism. Others include Schizosaccharomyces pombe (Beach and Nature. Nulure 25)o: 140 [1981]; EP 139.383 published May 2, 1995); Muyveromyces hosts (U.S. Pat. No. 4,943,529; Fleer et al., Bio/Technology, 9: 968-975 (1991) such as e.g., K lactis (MW98-8C. CBS683, CBS4574; Louvencourt et al. J. Bacteriof. 737 [1983]), K. fiagilis (ATCC 12,424), K. bulgaricus (ATCC 16,045), K wickeramii (ATCC 24,178), K waltii (ATCC 56,500), K. drosophilarum (ATCC 36.906); Van den Berg et al:, BioITechnoZogy 8: 135 (1990)); K. thermotoierans, and K. marxianus; yarrowia (EP 402.226); Pichia pastoris (EP 183,070); Sreekrishna et al., J. basic Microbial. 3: 265-278 [I 9881); Candid; Trichoderma reesia (EP 244,234); Neurospora crassa (Case et al., Proc. Natl. Acad Sci. USA 76: 5359-5263 [1979]); Schwanniomyces such as Schwanniomyces occidentulis (EP 394,538 published 3 Oct. 1, 1990); and filamentous fungi such as, e.g., Neurospora, Penicillium, Tolypocladium (WO 91100357 published Jan. 10, 19910, and Aspergillus hosts such as A. nidulans (Balance et al., Biachem. Biaphys. Res. Commun. 112: 284-289 [1983]; Tilbum et al., Gene 3: 205-221 [1983]; Yelton et al. Pmt. Natl. Acad. Sci. USA a: 1470-1474 [19841) and A. niger (Kelly and Hynes, EMBO. J. 4: 475-479 19851). Methylotropic yeasts are selected from the genera consisting of Hanscnuia. Candida, Kloeckera, Pichia. Saccharomyces. Torulopsis. and Rhodotoruia. A list of specific species that are exemplary of this class of yeast may be found in C. Antony, The Biochemistry of Methyiatrophs 269 (1982).

Suitable host cells for the expression of glycosylated HGF-AIh are derived from multicellular organisms. Examples of invertebrate cells include insect cells such as Drosophila S2 and Spodoptera Sp. Spodoptera high5 as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Viral., 36:59 (I 977)) Chinese hamster ovary cells/-DHFR(CHO, Urlaub and Chasin, Proc. Natl. Acad. Sci. USA, u:4216 (1980)); mouse sertoli cells (TM4, Mather, Biol. Reprod, 2:243-25 1 (1980)); human lung cells (W138. ATCC CCL 75); human liver cells (Hep G2, HB 8065); and mouse mammary tumor (MMT 060562, ATCC CCL51). The selection of the appropriate host cell is deemed to be within the skill in the art.

The nucleic acid (e.g., cDNA or genomic DNA) encoding the desired HGF-AIh polypeptide may be inserted into a replicable vector for cloning (amplification of the DNA) or for expression. Various vectors are publicly available. The vector may, for example, be in the form of a plasmid. cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The HGF-AIh polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector. or it may be a part of the HGF-AIh-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be. e.g., the yeast invertase leader, alpha factor leader or acid phosphatase leader, the C. albicans glucoamylase leader (EP 362,179 published Apr. 4, 1990), or the signal described in WO 9003646 published Nov. 15, 1990. In mammalian cell expression, mammalian signal sequences may be used to direct secretion of the protein, such as signal sequences from secreted polypeptides of the same or related species. as well as viral secretory leaders.

Both expression and cloning vectors contain a nucleic acid sequence that enables the vector to replicate in one or more selected host cells. Such sequences are well known for a variety of bacteria, yeast. and viruses. The origin of replication from the plasmid pBR322 is suitable for most Gram-negative bacteria, the 2u plasmid origin is suitable for yeast, and various viral origins (SV40, polyoma, adenovirus, VSV or BPV) are useful for cloning vectors in mammalian cells.

Expression and cloning vectors will typically contain a selection gene. also termed a selectable marker. TypicaI selection genes encode proteins that (a) confer resistance to antibiotics or other toxins. e.g., ampicillin, neomycin. methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO1031-or PRO1122-encoding nucleic acid, such as DHFR or thymidine kinase. An appropriate host cell when wild-type DHFR is employed is the CHO cell line deficient in DHFR activity. A suitable selection gene for use in yeast is the trpl gene present in the yeast plasmid YRp7 [Stinchcomb et al., Nature. 282:39 (1979); Kingsman et al., Gene, 7:141 (1979); Tschemper et al., Gene, 1J: 157 (1980)]. The trp1 gene provides a selection marker for a mutant strain of yeast lacking the ability to grow in tryptophan, for example, ATCC No. 44076 or PEPC1 [Jones, Genetics, a:12 (1977)].

Expression and cloning vectors usually contain a promoter operably linked to the PR01031- or PR01122-encoding nucleic acid sequence to direct mRNA synthesis. Promoters recognized by a variety of potential host cells are well known. Promoters suitable for use with prokaryotic hosts include the P-lactamase and lactose promoter systems [Goeddel et al., Nature, m:544 (1979)], alkaline phosphatase, a tryptophan (up) promoter system [Goeddel, Nucleic Acids Res. 8:4057 (1980); EP 36,776], and hybrid promoters such as the tat promoter. Promoters for use in bacterial systems also will contain a Shine-Dalgarno sequence operably linked to the DNA encoding the HGF-AIh polypeptide.

Examples of suitable promoting sequences for use with yeast hosts include the promoters for 3-phosphoglycerate kinase or other glycolytic enzymes such as enolase, glyceraldehydc-j-phosphate dehydrogenase, hexokinase, pyruvate decarboxylase, phosphofructokinase. glucose-6-phosphate isomerase, 3-phosphoglycerate mutase, pyruvate kinase,triosephosphate isomerase, phosphoglucose isomerase, and glucokinase.

Other yeast promoters. which are inducible promoters having the additional advantage of transcription controlled by growth conditions are the promoter regions for alcohol dehydrogenase 2, isocytochrome, acid phosphatase, degradative enzymes associated with nitrogen metabolism, metallothionein. glyceraldehyde-3-phosphate dehydrogenase, and enzymes responsible for maltose and galactose utilization. Suitable vectors and promoters for use in yeast expression are further described in EP 73.657. HGF-AIh transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter. and from heat-shock promoters, provided such promoters are compatible with the host cell systems.

Transcription of a DNA encoding an HGF-AIh polypeptide by higher eukaryotes may be increased by inserting an enhancer sequence into the vector. Enhancers are cis-acting elements of DNA. usually about from 10 to 300 bp, that act on a promoter to increase its transcription. Many enhancer sequences are now known from mammalian genes (globin, elastase, albumin. a-fetoprotein. and insulin). Typically, however, one will use an enhancer from a eukaryotic cell virus. Examples include the SV40 enhancer on the late side of the replication origin (bp 100-270). the cytomegalovirus early promoter enhancer. the polyoma enhancer on the late side of the replication origin, and adenovirus enhancers. The enhancer may be spliced into the vector at a position 5′ or 3′ to the HGF-AIh coding sequence, but is preferably located at a site 5′ from the promoter.

Expression vectors used in eukaryotic host cells will also contain sequences necessary for the termination of transcription and for stabilizing the mRNA. Such sequences are commonly available from the 5′ and. occasionally 3′, untranslated regions of eukaryotic or viral DNAs or cDNAs. These regions contain nucleotide segments transcribed as polyadenylated fragments in the untranslated portion of the mRNA encoding HGF-AIh.

Gene amplification and/or expression may be measured in a sample directly, for example, by conventional Southern blotting, Northern blotting to quantitate the transcription of mRNA, dot blotting (DNA analysis), or in situ hybridization, using an appropriately labeled probe, based on the sequences provided herein. Alternatively, antibodies may be employed that can recognize specific duplexes. including DNA duplexes, RNA duplexes. and DNA-RNA hybrid duplexes or DNA-protein duplexes. The antibodies in turn may be labeled and the assay may be carried out where the duplex is bound to a surface. so that upon the formation of duplex on the surface. the presence of antibody bound to the duplex can be detected.

Gene expression, alternatively may be measured by immunological methods, such as immunohistochemical staining of cells or tissue sections and assay of cell culture or body fluids, to quantitate directly the expression of gene product. Antibodies useful for innnunohistochemical staining and/or assay of sample fluids may be either monoclonal or polyclonal, and may be prepared in any mammal. Conveniently, the antibodies may be prepared against a native sequence HGF-AIh polypeptide or against a synthetic peptide based on the DNA sequences provided herein or against exogenous sequence fused to HGF-AIh-encoding DNA and encoding a specific antibody epitope.

Forms of HGF-AIh may be recovered from culture medium or from host cell lysates. If membrane-bound, it can be released from the membrane using a suitable detergent solution or by enzymatic cleavage. Cells employed in expression of HGF-AIh polypeptides can be disrupted by various physical or chemical means. such as freeze-thaw cycling, sonication. mechanical disruption, or cell lysing agents. It may be desired to purify HGF-AIh from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using. for example, Sephadex® G-75; protein A Sepharose® columns to remove contaminants such as IgG; and metal chelating columns to bind epitope-tagged forms of the HGF-AIh polypeptide. Various methods of protein purification may be employed and such methods are known in the art. The purification step selected will depend, for example, on the nature of the production process used and the particular HGF-AIh peptide produced.

The present invention further provides anti-HGF-AIh polypeptide antibodies. Exemplary antibodies include polyclonal, monoclonal. humanized, bispecifc, and heteroconjugate antibodies. The anti-HGF-AIh antibodies of the present invention may comprise polyclonal antibodies. Methods of preparing polyclonal antibodies are known to the skilled artisan. Polyclonal antibodies can be raised in a mammal. for example, by one or more injections of an immunizing agent and, if desired, an adjuvant. Typically, the immunizing agent and/or adjuvant will be injected in the mammal by multiple subcutaneous or intraperitoneal injections. The immunizing agent may include the HGF-AIh polypeptide or a fusion protein thereof. It may be useful to conjugate the immunizing agent to a protein known to be immunogenic in the mammal being immunized. Examples of such immunogenic proteins include but are not limited to keyhole limpet hemocyanin. serum albumin, bovine thyroglobulin. and soybean trypsin inhibitor. Examples of adjuvants which may be employed include Freund's complete adjuvant monophosphoryl Lipid A synthetic trehalose dicorynomycolate. The immunization protocol may be selected by one skilled in the art without undue experimentation.

The anti-HGF-AIh antibodies may, alternatively, be monoclonal antibodies. Monoclonal antibodies may be prepared using hybridoma methods, such as those described by Kohler and Milstein. In a hybridoma method, a mouse, hamster, or other appropriate host animal is typically immunized with an immunizing agent to elicit B lymphocytes that produce or are capable of producing antibodies that will specifically bind to the immunizing agent.

The immunizing agent will typically include the HGF-AIh polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol to form a hybridoma cell. Immortalized cell lines are usually transformed mammalian cells, particularly myeloma cells of rodent, bovine and human origin. Usually rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

The culture medium in which the hybridoma cells are cultured can then be assayed for the presence of monoclonal antibodies directed against an HGF-AIh polypeptide. Preferably, the binding specificity of monoclonal antibodies produced by the hybridoma cells is determined by immunoprecipitation or by an in vitro binding assay, such as radioimmunoassay (R1A) or enzyme-linked immunoabsorbent assay (ELISA). Such techniques and assays are known in the art. The binding affinity of the monoclonal antibody can, for example, be determined by the Scatchard analysis.

The monoclonal antibodies secreted by the subclones may be isolated or purified from the culture medium or ascites fluid by conventional immunoglobulin purification procedures such as, for example, protein A-Sepharose®, hydroxylapatite chromatography, gel electrophoresis, dialysis, or affinity chromatography.

The monoclonal antibodies may also be made by recombinant DNA methods such as those described, for example, in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells. Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

The anti-HGF-AIh antibodies of the present invention may further comprise humanized antibodies or human antibodies. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers by substituting rodent CDRs or CDR sequences for the corresponding sequences of a human antibody.

Human antibodies can also be produced using various techniques known in the art, including phage display libraries [Hoogenboom and Winter, J. Mol. BioZ., 227:381 (1991); Marks et al, J. Mol. Biol., m:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)]. Similarly, human antibodies can be made by introducing human immunoglobulin loci into transgenic animals, e.g. mice in which the endogenous immunoglobulin genes have been partially or complete inactivated. Upon challenge, human antibody production is observed which closely resembles that seen in humans in all respects, including gene rearrangement, assembly and antibody repertoire.

The antibodies of the present invention may also be used in ADEPT by conjugating the antibody to a prodrug-activating enzyme which converts a prodrug (e.g. a peptidyl chemotherapeutic agent, see WO 81/01145) to an active anti-cancer drug. See. for example, WO 88/07378 and U.S. Pat. No. 4,975,278. The enzyme component of the immunoconjugate useful for ADEPT includes any enzyme capable of acting on a prodrug in such as way so as to convert it into its more active, cytotoxic form.

The enzymes of this invention can be covalently bound to the anti-HGF-AIh antibodies by techniques well known in the art such as the use of the heterobifunctional cross-linking agents discussed above. Alternatively, fusion proteins comprising at least the antigen binding region of the antibody of the invention linked to at least a functionally active portion of an enzyme of the invention can be constructed using recombinant DNA techniques well known in the art (see, e.g. Neuberger et al., Nature 312: 604-608 (1984)).

Bispecific anti-HGF-AIh antibodies area further aspect of the invention. Bispecific antibodies are monoclonal, preferably human or humanized, antibodies that have binding specificities for at least two different antigens. In the present case, one of the binding specificities is for HGF-AIh, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities. Because of the random assortment of immunoglobulin heavy and light chains, these hybridomas (quadromas) produce a potential mixture of ten different antibody molecules of which only one has the correct bispecific structure. The purification of the correct molecule is usually accomplished by routine affinity chromatography steps.

Antibody variable domains with the desired binding specificities (antibody-antigen combining sites) can be fused to immunoglobulin constant domain sequences. The fusion preferably is with an immunoglobulin heavy-chain constant domain. comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism.

Bispecific antibodies can be prepared as full length antibodies or antibody fragments. Techniques for generating bispecific antibodies from antibody fragments have been described in the literature.

Antibodies with more than two valencies are contemplated. For example, trispecific antibodies can be prepared. Tutt et al., J Immunol. 147: 60 (1991).

Heteroconjugate antibodies are also within the scope of the present invention. Heteroconjugate antibodies are composed of two covalently joined antibodies. Such antibodies have, for example, been proposed to target immune system cells to unwanted cells, and for treatment of HIV infection. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

The invention also pertains to immunoconjugates comprising an antibody conjugated to a cytotoxic agent such as a chemotherapeutic agent, toxin (e.g. an enzymatically active toxin of bacterial, fungal, plant or animal origin, or fragments thereof, or a small molecule toxin), or a radioactive isotope (i.e., a radioconjugate).

In another embodiment, the antibody may be conjugated to a “receptor” (such streptavidin) for utihzation in tumor pretargeting wherein the antibody-receptor conjugate is administered to the patient, followed by removal of unbound conjugate from the circulation using a clearing agent and then administration of a “ligand” (e-g. avidin) which is conjugated to a cytotoxic agent (e.g. a radionucleotide).

The antibodies disclosed herein may also be formulated as immunoliposomes to enhance serum half life. Liposomes containing the antibody are prepared by methods known in the art. Liposomes can be generated by the reverse phase evaporation method with a lipid composition comprising phosphatidylcholine, cholesterol and PEG derivatized phosphatidylethanolamine (PEG-PE). Liposomes are extruded through filters of defined pore size to yield liposomes with the desired diameter. Fab′ fragments of the antibody of the present invention can be conjugated to the liposomes via a disulfide interchange reaction. A chemotherapeutic agent may optionally be included within the liposome.

Antibodies specifically binding an HGF-AIh polypeptide identified herein, as well as other molecules identified by the screening assays disclosed hereinbefore, can be administered for the treatment of various disorders in the form of pharmaceutical compositions. If an HGF-AIh polypeptide is intracellular and whole antibodies are used as inhibitors, internalizing antibodies are preferred. However, lipofections or liposomes can also be used to deliver the antibody, or an antibody fragment into cells. Where antibody fragments are used, the smallest inhibitory fragment that specifically binds to the binding domain of the target protein is preferred. For example, based upon the variable-region sequences of an antibody. peptide molecules can be designed that retain the ability to bind the target protein sequence. Such peptides can be synthesized chemically and/or produced by recombinant DNA technology. The formulation herein may also contain more than one active compound as necessary for the particular indication being treated. preferably those with complementary activities that do not adversely affect each other. Alternatively, or in addition, the composition may comprise an agent that enhances its function, such as, for example, a cytotoxic agent, cytokines, chemotherapeutic agent, or growth-inhibitory agent. Such molecules are suitable present in combination in amounts that are effective for the purpose intended. The active ingredients may also be entrapped in microcapsules prepared, for example, by coascervation techniques or by interfacial polymerization, for example, hydroxymethylcellulose or felatin-microcapsules and poly-(methylmethactylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes. Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid y-ethyl-L-glutamate, non-degradable ethylene-vinylacetate, degradable lactic acid-glycolic acid copolymers such as the LUPRON DEPOT™ (injectable microspheres composed of lactic acid-glycolic acid copolymer and leuprolide acetate), and poly-D-(−)3-hydroxylbutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanisms involved. For example, if the aggregation mechanism is discovered to be intermolecular S—S bond formation through thiosuifide interchange, stabilization may be achieved by modifying sulfhydryl residues, lyophilizing from acidic solutions, controlling moisture content, using appropriate additives, and developing specific polymer matrix compositions.

Anti-HGF-AIh antibodies and fragments are useful for a variety of purposes including but not limited to the affinity purification of HGF-AIh polypeptides from recombinant cell culture or natural sources. In this process, the antibodies against an HGF-AIh polypeptide are immobilized on a suitable support, such a Sephadex® resin or filter paper, using methods well known in the art. The immobilized antibody then is contacted with a sample containing the HGF-AIh polypeptide to be purified, and thereafter the support is washed with a suitable solvent that will remove substantially all the material in the sample except the HGF-AIh polypeptide which is bound to the immobilized antibody. Finally, the support is washed with another suitable solvent that will release the HGF-AIh polypeptide from the antibody.

In another embodiment, the invention encompasses methods of screening compounds to identity those that mimic the HGF-AIh (agonists) or prevent the effect of the HGF-AIh (antagonists). Screening assays for antagonist drug candidates are designed to identity compounds that bind or complex with the HGF-AIh encoded by the genes identified herein. or otherwise interfere with the interaction of the encoded polypeptides with other cellular proteins. Such screening assays will include assays amenable to high-throughput screening of chemical libraries, making them particularly suitable for identifying small molecule drug candidates.

The assays can be performed in a variety of formats including protein-protein binding assays, biochemical screening assays, immunoassays, and cell-based assays, which are well characterized in the art. In binding assays, the interaction is binding and the complex formed can be isolated or detected in the reaction mixture. In a particular embodiment, the HGF-AIh polypeptide encoded by the gene identified herein or the drug candidate is immobilized on a solid phase, e.g., on a microtiter plate, by covalent or non-covalent attachments. Noncovalent attachment generally is accomplished by coating the solid surface with a solution of the HGF-AIh polypeptide and drying. Alternatively, an immobilized antibody, e.g., a monoclonal antibody, specific for the HGF-AIh polypeptide to be immobilized can be used to anchor it to solid surface. The assay is performed by adding the non-immobilized component, which may be labeled by a detectable label, to the immobilized component, which may be labeled by a detectable label to the immobilized component, e.g., the coated surface containing the anchored component. When the reaction is complete. the non-reacted components are removed, e.g., by washing, and complexes anchored on the solid surface are detected. When the originally non-immobilized component carries a detectable label. the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular HGF-AIh polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored through gradients or chromatographic columns. In addition, protein-protein interactions can be monitored by using a yeast-based genetic system described by Fields and co-workers. Many transcriptional activators, such as yeast GAL4. consist of two physically discrete modular domains, one acting as the DNA-binding domain, while the other functions as the transcription-activation domain. The yeast expression system described in the foregoing publications (generally referred to as the “two-hybrid system”). takes advantage of this property, and employs two hybrid proteins, one in which the target protein is fused to the DNA-binding domain of GAL4, and another, in which candidate activating proteins are fused to the activation domain. The expression of GAL1-lacZ reporter gene under control of a GAL4activated promoter depends on reconstitution of GAL4 activity via protein-protein interaction. Colonies containing interacting polypeptide are detected with chromogenic substrate for /3-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding an HGF-AIh polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound, In addition, a placebo may be added to a third reaction mixture. to serve as a positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

Antagonists may be detected by combining the HGF-AIh polypeptide and a potential antagonist with membrane-bound HGF-AIh polypeptide receptors or recombinant receptors under appropriate conditions for a competitive inhibition assay. The HGF-AIh polypeptide can be labeled, such as by radioactivity, such that the number of HGF-AIh polypeptide molecules bound to the receptor can be used to determine the effectiveness of the potential antagonist. The gene encoding the receptor can be identified by numerous methods known to those of skill in the art, for example, ligand panning and FACS sorting. Preferably, expression cloning is employed wherein polyadenylated RNA is prepared from a cell responsive to the HGF-AIh polypeptide and a cDNA library created from this RNA is divided into pools and used to transfect COS cells or other cells that are not responsive to the HGF-AIh polypeptide. respectively. Transfected cells that are grown on glass slides are exposed to labeled HGF-AIh. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled HGF-AIh can be photoaffinity-linked with cell membrane or extract preparations that express the receptor molecule. Cross-linked material is resolved by PAGE and exposed to X-ray film. The labeled complex containing the receptor can be excised. resolved into peptide fragments, and subjected to protein micro-sequencing. The amino acid sequence obtained from micro-sequencing would be used to design a set of degenerate oligonucleotide probes to screen a cDNA library to identity the gene encoding the putative receptor.

In another assay for antagonists. mammalian cells or a membrane preparation expressing the receptor would be incubated with labeled HGF-AIh in the presence of the candidate compound. The ability of the compound to enhance or block this interaction could then be removed.

Another potential HGF-AIh antagonist is an antisense RNA or DNA construct prepared using antisense technology, where, e.g., an antisense RNA or DNA molecule acts to block directly the translation of mRNA by hybridizing to targeted mRNA and preventing its translation into protein. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature HGF-Aih, is used to design an antisense RNA oligonucleotide sequence of about 10 to 40 base pairs in length. A DNA oligonucleotide is designed to be complementary to a region of the gene involved in transcription thereby preventing transcription and the production of the HGF-AIh polypeptide. The antisense RNA oligonucleotide hybridizes to the mRNA in vivo and blocks translation of the mRNA molecule into the HGF-AIh. The oligonucleotides described above can also be delivered to cells such that the antisense RNA or DNA may be expressed in vivo to inhibit production of the HGF-AIh. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the HGF-AIh polypeptide, thereby blocking the normal biological activity of the HGF-AIh. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonuclytic cleavage. Specific ribozyme cleavage sites within a potential RNA target can be identified by known techniques. Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex.

Another use of the compounds of the invention described herein is to help diagnose whether a disorder is driven, to some extent, by HGF-AIh modulated signaling. A diagnostic assay to determine whether a particular disorder is driven by HGF-AIh signaling, can be carried out using the following steps: (1) culturing test cells or tissues expressing HGF-AIh; (2) administering a compound which can inhibit HGF-AIh modulated signaling; and (3) measuring the HGF-AIh mediated phenotypic effects in the test cells. The steps can be carried out using standard techniques in light of the present disclosure. For example, standard techniques can be used to isolate cells or tissues and culturing or in vivo. Compounds of varying degree of selectivity are useful for diagnosing the role of HGF-AIh. For example, compounds which HGF-AIh in addition to another form of adaptor molecule can be used as an initial test compound to determine if one of several adaptor molecules drive the disorder. The selective compounds can then be used to further eliminate the possible role of the other adaptor proteins in driving the disorder. Test compounds should be more potent in inhibiting intracellular signaling activity than in exerting a cytotoxic effect. The IC50 and LD50 can be measured by standard techniques. such as an MTT assay, or by measuring the amount of LDH released. The degree of IC50 & LD50 of a compound should be taken into account in evaluating the diagnostic assay. Generally, the larger the ratio the more relative the information. Appropriate controls take into account the possible cytotoxic effect of a compound of a compound, such as treating cells not associated with a cell proliferative disorder with a test compound, can also be used as part of the diagnostic assay. The diagnostic methods of the invention involve the screening for agents that modulate the effects of HGF-AIh upon cell proliferative disorders.

For example, antibodies, including antibody fragments, can be used to qualitatively or quantitatively detect the expression of proteins encoded by the disease-related genes (“marker gene products”). The antibody preferably is equipped with a detectable, e.g. fluorescent, label, and binding can be monitored by light microscopy, flow cytometry, fluorimetry, or other techniques known in the art.

In situ detection of antibody binding to the marker gene products can be performed, for example, by immunofluorescence or immunoelectron microscopy. For this purpose, a histological specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

The HGF-AIh, antagonists or agonists thereof (e.g., antibodies), as well as other molecules identified by the screening assays disclosed hereinbefore, can be employed as therapeutic agents. Such therapeutic agents are formulated according to known methods to prepare pharmaceutically useful compositions, whereby the HGF-AIh, antagonist or agonist thereof is combined in admixture with a pharmaceutically acceptable carrier.

In the case of HGF-AIh antagonist or agonist antibodies, if the protein encoded by the amplified gene is intracellular and antibodies are used as inhibitors, internalizing antibodies are preferred. However, lipofections or liposomes can also be used to deliver the antibody, or an antibody fragment, into cells. Where antibody fragments are used. the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology).

Therapeutic formulations are prepared for storage by mixing the active ingredient having the desired degree of purity with optional pharmaceutically acceptable carriers, excipients or stabilizers in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride. benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin. or immunoglobulins; hydrophilic polymers such as polyvinylpyrrolidone; amino acids such as glycine, glutamine, asparagine, histidine, arginine, or lysine; monosaccharides, disaccharides. and other carbohydrates including glucose. mannose, or dextrins; chelating agents such as EDTA: sugars such as sucrose. mannitol. trehalose or sorbitol; salt-forming counter-ions such as sodium; metal complexes (e.g. Zn-protein complexes); and/or non-ionic surfactants such as TWEEN™, PLURONIC™ or polyethylene glycol (PEG).

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active ingredients may also be entrapped in microcapsules prepared, for example, by coacervation techniques or by interfacial polymerization. for example, hydroxymethylcellulose or gelatin-microcapsules and poly-(methylmethacylate) microcapsules, respectively, in colloidal drug delivery systems (for example, liposomes, albumin microspheres.microemulsions, nano-particles and nanocapsules) or in macroemulsions.

The formulations to be used for in viva administration must be sterile. This is readily accomplished by filtration through sterile filtration membranes. Therapeutic compositions herein generally are placed into a container having a sterile access port, for example, and intravenous solution bag or vial having a stopper pierceable by a hypodermic injection needle.

Sustained-release formulations may be developed using poly lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition.

While polymers such as ethylene-vinyl acetate and lactic acid-glycolic acid enable release of molecules for over 100 days, certain hydrogels release proteins for shorter time periods. When encapsulated antibodies remain in the body for a long time. they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiodisulfide interchange, stabilization may be achieved by modifying sulfhydryl residues, lyophilizing from acidic solutions. controlling moisture content. using appropriate additives: and developing specific polymer matrix compositions.

The active agents of the present invention are administered to a mammal, preferably a human, in accord with known methods, such as intravenous administration as a bolus or by continuous infusion over a period of time, by intramuscular, intraperitoneal, intracerebral, intracerobrospinal, subcutaneous. intra-articular, intrasynovial, intrathecal, intraoccular, intralesional, oral, topical. inhalation or through sustained release.

Other therapeutic regimens may be combined with the administration of the HGF-AIh, antagonists or antagonists, anti-cancer agents. For the prevention or treatment of disease, the appropriate dosage of an active agent will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective does for human therapy.

In another embodiment of the invention. an article of manufacture containing materials useful for the diagnosis or treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is typically an HGF-AIh polypeptide. antagonist. or agonist thereof. The label on. or associated with, the container indicates that the composition is used for diagnosing or treating the condition of choice. The article of manufacture may further comprise a second container comprising a pharmaceutically-acceptable buffer. such as phosphate-buffered saline. Ringer's solution and dextrose solution. It may further include other materials desirable from a commercial and user standpoint, including other buffers, diluents, filters, needles, syringes, and package inserts with instructions for use.

The following examples are offered for illustrative purposes only, and are not intended to limit the scope of the present invention in any way.

### EXAMPLE 1

### Expression and Purification of an HGF-AIh in E. coli

The bacterial expression vector pQE60 is used for bacterial expression in this example. (QIAGEN, Inc., Chatsworth, Calif.). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., and suitable single restriction enzyme cleavage sites. These elements are arranged such that a DNA fragment encoding a polypeptide can be inserted in such a way as to produce that polypeptide with the six His residues (i.e., a “6×His tag”) covalently linked to the carboxyl terminus of that polypeptide. However, a polypeptide coding sequence can optionally be inserted such that translation of the six His codons is prevented and, therefore, a polypeptide is produced with no 6×His tag.

The nucleic acid sequence encoding the desired portion of an HGF-AIh lacking the hydrophobic leader sequence is amplified from a cDNA clone using PCR oligonucleotide primers (based on the nucleic acid sequences presented, e.g., as presented in SEQ ID NO: 1 or SEQ ID NO: 3), which anneal to the amino terminal encoding DNA sequences of the desired portion of an HGF-AIh and to sequences in the construct 3′ to the cDNA coding sequence. Additional nucleotides containing restriction sites to facilitate cloning in the pQE60 vector are added to the 5′ and 3′ sequences, respectively.

For cloning an HGF-AIh, the 5′ and 3′ primers have nucleotides corresponding or complementary to a portion of the coding sequence of an HGF-AIh, e.g., as presented in SEQ ID NO: 1 or in SEQ ID NO: 3, according to known method steps. One of ordinary skill in the art would appreciate, of course, that the point in a polypeptide coding sequence where the 5′ primer begins can be varied to amplify a desired portion of the complete polypeptide shorter or longer than the mature form.

The amplified HGF-AIh nucleic acid fragments and the vector pQE60 are digested with appropriate restriction enzymes and the digested DNAs are then ligated together. Insertion of the HGF-AIh DNA into the restricted pQE60 vector places an HGF-AIh polypeptide coding region including its associated stop codon downstream from the IPTG-inducible promoter and in-frame with an initiating AUG codon. The associated stop codon prevents translation of the six histidine codons downstream of the insertion point.

The ligation mixture is transformed into competent E. coli cells using standard procedures such as those described in Sambrook, et al., 1989; Ausubel, 1987-1998. E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing HGF-AIh polypeptide, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6M guanidine-HCl, pH 8. The cell debris is removed by centrifugation, and the supernatant containing the HGF-AIh is dialyzed against 50 mM Na-acetate buffer pH 6, supplemented with 200 mM NaCl. Alternatively, a polypeptide can be successfully refolded by dialyzing it against 500 mM NaCl, 20% glycerol, 25 mM Tris/HCl pH 7.4, containing protease inhibitors.

If insoluble protein is generated, the protein is made soluble according to known method steps. After renaturation the polypeptide is purified by ion exchange, hydrophobic interaction and size exclusion chromatography. Alternatively, an affinity chromatography step such as an antibody column is used to obtain pure HGF-AIh. The purified polypeptide is stored at 4° C. or frozen at −40° C. to −120° C.

### EXAMPLE 2

### Cloning and Expression of an HGF-AIh Polypeptide in a Baculovirus Expression System

In this example, the plasmid shuttle vector pA2 GP is used to insert the cloned DNA encoding the mature polypeptide into a baculovirus to express an HGF-AIh, using a baculovirus leader and standard methods as described in Summers, et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by the secretory signal peptide (leader) of the baculovirus gp67 polypeptide and convenient restriction sites such as BamHI, Xba I, and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Other baculovirus vectors are used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow, et al., Virology 170:31-39.

The cDNA sequence encoding the mature HGF-AIh polypeptide in a clone, lacking the AUG initiation codon and the naturally associated nucleotide binding site, is amplified using PCR oligonucleotide primers corresponding to the 5′ and 3′ sequences of the gene. Non-limiting examples include 5′ and 3′ primers having nucleotides corresponding or complementary to a portion of the coding sequence of an HGF-AIh polypeptide, e.g., as presented in SEQ ID NO: 1 or 3, according to known method steps.

The amplified fragment is isolated from a 1% agarose gel using a commercially available kit (e.g., “Geneclean,” BIO 101 Inc., La Jolla, Calif.). The fragment then is then digested with the appropriate restriction enzyme and again is purified on a 1% agarose gel. This fragment is designated herein “F1”.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid V1 are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria are identified that contain the plasmid with the human HGF-AIh gene using the PCR method, in which one of the primers that is used to amplify the gene and the second primer is from well within the vector so that only those bacterial colonies containing the HGF-AIh gene fragment will show amplification of the DNA. The sequence of the cloned fragment is confirmed by DNA sequencing. This plasmid is designated herein pBac HGF-AIh.

Five μg of the plasmid pBacHGF-AIh is co-transfected with 1.0 μg of a commercially available linearized baculovirus DNA (“BaculoGold™ baculovirus DNA”, Pharmingen, San Diego, Calif.), using the lipofection method described by Felgner, et al., Proc. Natl. Acad. Sci. USA 84:7413-7417 (1987). 1 μg of BaculoGold™ virus DNA and 5 μg of the plasmid pBac HGF-AIh are mixed in a sterile well of a microtiter plate containing 50 μl of serum-free Grace's medium (Life Technologies, Inc., Rockville, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed, according to known methods. An agarose gel with “Blue Gal” (Life Technologies, Inc., Rockville, Md.) is used to allow easy identification and isolation of gal-expressing clones, which produce blue-stained plaques. (A detailed description of a “plaque assay” of this type can also be found in the user's guide for insect cell culture and baculovirology distributed by Life Technologies, Inc., Rockville, Md., page 9-10). After appropriate incubation, blue stained plaques are picked with a micropipettor tip (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-HGF-AIh.

To verify the expression of the HGF-AIh gene, Sf9 cells are grown in Grace's medium supplemented with 10% heat-inactivated FBS. The cells are infected with the recombinant baculovirus V-HGF-AIh at a multiplicity of infection (“MOI”) of about 2. Six hours later the medium is removed and is replaced with SF900 II medium minus methionine and cysteine (available, e.g., from Life Technologies, Inc., Rockville, Md.). If radiolabeled polypeptides are desired, 42 hours later, 5 mCi of 35S-methionine and 5 mCi 35S-cysteine (available from Amersham) are added. The cells are further incubated for 16 hours and then they are harvested by centrifugation. The polypeptides in the supernatant as well as the intracellular polypeptides are analyzed by SDS-PAGE followed by autoradiography (if radiolabeled). Microsequencing of the amino acid sequence of the amino terminus of purified polypeptide can be used to determine the amino terminal sequence of the mature polypeptide and thus the cleavage point and length of the secretory signal peptide.

### EXAMPLE 3

### Cloning and Expression of HGF-AIh in Mammalian Cells

A typical mammalian expression vector contains at least one promoter element, which mediates the initiation of transcription of mRNA, the polypeptide coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or PLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded polypeptide. The DHFR (dihydrofolate reductase) marker is useful to develop cell lines that carry several hundred or even several thousand copies of the gene of interest. Another useful selection marker is the enzyme glutamine synthase (GS) (Murphy, et. al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pHGF-AIh HA, is made by cloning a cDNA encoding HGF-AIh into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eucaryotic cells; (4) a CMV promoter, a polylinker, an SV40 intron; (5) several codons encoding a hemagglutinin fragment (i.e., an “HA” tag to facilitate purification) or HIS tag (see, e.g, Ausubel, supra) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin polypeptide described by Wilson, et al., Cell 37:767-778 (1984). The fusion of the HA tag to the target polypeptide allows easy detection and recovery of the recombinant polypeptide with an antibody that recognizes the HA epitope. pcDNAIII contains, in addition, the selectable neomycin marker.

A DNA fragment encoding the HGF-AIh is cloned into the polylinker region of the vector so that recombinant polypeptide expression is directed by the CMV promoter. The plasmid construction strategy is as follows. The HGF-AIh cDNA of a clone is amplified using primers that contain convenient restriction sites, much as described above for construction of vectors for expression of HGF-AIh in E. coli. Non-limiting examples of suitable primers include those based on the coding sequence presented in SEQ ID NO: 1 or in SEQ ID NO: 3, as they encode an HGF-Aih polypeptide as described herein.

The PCR amplified DNA fragment and the vector, pcDNAI/Amp, are digested with suitable restriction enzyme(s) and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the HGF-AIh-encoding fragment.

For expression of recombinant HGF-AIh, COS cells are transfected with an expression vector, as described above, using DEAE-DEXTRAN, as described, for instance, in Sambrook, et al., Molecular Cloning: a Laboratory Manual, Cold Spring Laboratory Press, Cold Spring Harbor, N.Y. (1989). Cells are incubated under conditions for expression of HGF-AIh by the vector.

Expression of the HGF-AIh-HA fusion polypeptide is detected by radiolabeling and immunoprecipitation, using methods described in, for example Harlow, et al., Antibodies: A Laboratory Manual, 2nd Ed., Cold Spring Harbor Laboratory Press, Cold Spring Harbor, N.Y. (1988). To this end, two days after transfection, the cells are labeled by incubation in media containing 35S-cysteine for 8 hours. The cells and the media are collected, and the cells are washed and lysed with detergent-containing RIPA buffer: 150 mM NaCl, 1% NP-40, 0.1% SDS; 0.5% DOC, 50 mM TRIS, pH 7.5, as described by Wilson, et al. cited above. Proteins are precipitated from the cell lysate and from the culture media using an HA-specific monoclonal antibody. The precipitated polypeptides then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of HGF-AIh polypeptide. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the HGF-AIh in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete HGF-AIh polypeptide is amplified using PCR oligonucleotide primers corresponding to the 5′ and 3′ sequences of the gene. Non-limiting examples include 5′ and 3′ primers having nucleotides corresponding or complementary to a portion of the coding sequence of an HGF-AIh, e.g., as presented in SEQ ID NO: 1 or in SEQ ID NO: 3, according to known method steps.

The amplified fragment is digested with suitable endonucleases and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations. of methotrexate (1 mM, 2 mM, 5 mM, 10 mM, 20 mM). The same procedure is repeated until clones are obtained which grow at a concentration of 100-200 mM. Expression of the desired gene product is analyzed, for instance, by SDS-PAGE and Western blot or by reverse phase HPLC analysis.

### EXAMPLE 4

### Tissue Distribution of HGF-AIh mRNA Expression

Northern blot analysis is carried out to examine HGF-AIh gene expression in human tissues, using methods described by, among others, Sambrook, et al., cited above. A cDNA probe containing nucleotide sequence encoding an HGF-AIh polypeptide (such as, e.g., SEQ ID NO: 2 or SEQ ID NO: 4) is labeled with ³²P using the Rediprime™ DNA labeling system (Amersham Life Science), according to the manufacturer's instructions. After labeling, the probe is purified using a CHROMA SPIN-100™ column (Clontech Laboratories, Inc.), according to the manufacturer's protocol number PT1200-1. The purified and labeled probe is used to examine various human tissues for HGF-AIh mRNA. Multiple Tissue Northern (MTN) blots containing various human tissues (H) or human immune system tissues (IM) are obtained from Clontech and are examined with the labeled probe using ExpressHyb hybridization solution (Clontech) according to manufacturer's protocol number PT1190-1. Following hybridization and washing, the blots are mounted and exposed to film at −70° C. overnight, and films developed according to standard procedures. The results show HGF-AIh polypeptides to be selectively expressed in other tissues.

What is claimed is:

1. Isolated nucleic acid comprising DNA having at least an 80% sequence identity to (a) a polynucleotide sequence encoding a polypeptide comprising amino acid residues 1 through 245, inclusive of SEQ ID NO: 2, (b) the complement of the DNA molecule of (a), (c) a polynucleotide sequence encoding a polypeptide comprising amino acid residues from about 1 or from about 37 to about 500 inclusive, of SEQ ID NO: 4, and (d) the complement of the DNA molecule of (c).

2. The nucleic acid of claim 1, wherein said DNA comprises (a) from about 145 to about 879 of SEQ ID NO: 1, or (b) from about 147 or from about 258 to about 1647 of SEQ ID NO: 3.

3. The nucleic acid of claim 1, wherein said DNA comprises (a) the mature polypeptide coding portion of SEQ ID NO: 1 or, (b) the mature polypeptide coding portion of SEQ ID NO: 3.

4. The isolated nucleic acid molecule of claim 1 that encodes a polypeptide comprising: (a) residues from 1 to about 245 of SEQ ID NO: 2; (b) residues from about 1 to about 500 inclusive, of SEQ ID NO: 4; or (c) residues from about 37 to about 500 of SEQ ID NO: 4.

5. An isolated nucleic acid molecule encoding an HGF-AIh polypeptide, wherein said nucleic acid molecule hybridizes to the complement of a nucleic acid sequence that encodes: (a) amino acids 1 to about 245 of SEQ ID NO: 2; (b) residues from about 1 to about 500 inclusive, of SEQ ID NO: 4; (c) residues from about 37 to about 500 of SEQ ID NO: 4; or (d) fragments of (a), (b), or (c), wherein said fragments are sufficient to provide a binding site for an anti-HGF-AIh antibody.

6. The isolated nucleic acid molecule of claim 5, wherein said hybridization occurs under stringent conditions.

7. An isolated nucleic acid molecule comprising (a) DNA encoding a polypeptide scoring at least 80% positives when compared to the sequence of amino acid residues selected from: (a) from 1 to about 245, inclusive, of SEQ ID NO: 2; (b) the complement of the DNA of (a); (c) from about 1 to about 500 inclusive of SEQ ID NO: 4, or from about 37 to about 500 inclusive of SEQ ID NO: 4; or (d) the complement of the DNA of (c).

8. An isolated nucleic acid molecule at least about 250 nucleotides in length (or its complement) that is produced by:
(a) hybridizing a first nucleic acid molecule under stringent hybridization conditions with a second nucleic acid molecule and,
(b) isolating said first nucleic acid molecule; wherein said second nucleic acid molecule comprises sequence encoding a polypeptide comprising:
(i) from 1 to about 245, inclusive of SEQ ID NO: 2, or
(ii) from about 1 or from about 37 to about 500, inclusive of SEQ ID NO: 4.

9. The isolated nucleic acid molecule of claim 8, which has at least about 80% sequence identity to the mature coding portion of SEQ ID NO: 1 or SEQ ID NO: 3.

10. A vector comprising the nucleic acid molecule of any of claims 1 to 9.

11. The vector of claim 10, wherein said nucleic acid molecule is operably linked to control sequences recognized by a host cell transformed with a vector.

12. A host cell comprising the vector of claim 11.

13. The host cell of claim 12, wherein said cell is a CHO cell.

14. The host cell of claim 12, wherein said cell is an E. coli.

15. The host cell of claim 12, wherein said cell is a yeast cell.

16. A process for producing an HGF-AIh polypeptide comprising culturing the host cell of claim 12 under conditions suitable for expression of said HGF-AIh polypeptide and recovering said HGF-AIh polypeptide from said culture.

17. An isolated polypeptide encoding an amino acid sequence having at least about 80% sequence identity to the sequence comprising (a) residues 1 to about 245 of SEQ ID NO: 2; or (b) residues from about 37 to about 500 or SEQ ID NO: 4.

18. The isolated HGF-AIh polypeptide of claim 17 comprising: (a) amino acid residues 1 to about 245 of SEQ ID NO: 2; or (b) amino acid residues from about 37 to about 500 of SEQ ID NO: 4.

19. An isolated HGF-AIh polypeptide scoring at least 80% positives when compared to: (a) amino acid residues from about 1 to about 245 of SEQ ID NO: 2; or (b) amino acid residues from about 37 to about 500 of SEQ ID NO: 4.

20. An isolated HGF-AIh polypeptide comprising: (a) the sequence of amino acid residues from about 1 to about 245 of SEQ ID NO: 2, or a fragment thereof sufficient to provide a binding site for an anti-HGF-AIh antibody; or (b) from about 37 to about 500 of SEQ ID NO: 4, or a fragment thereof sufficient to provide a binding site for an anti-HGF-AIh antibody.

21. An isolated polypeptide produced by (i) hybridizing a test DNA molecule under stringent conditions with (a) a DNA molecule encoding a polypeptide comprising amino acid residues from about 1 to about 245 of SEQ ID NO: 2, or amino acid residues from about 37 to about 500 of SEQ ID NO: 4; or (b) the complement of the DNA molecule of (a); (ii) culturing a host cell comprising said test DNA molecule under conditions suitable for the expression of said isolated polypeptide, and (iii) recovering said isolated polypeptide from said cell culture.

22. The isolated polypeptide of claim 21, wherein said test DNA has at least about 80% sequence identity to (a) or (b).

23. A chimeric molecule comprising a polypeptide fragment of SEQ ID NO: 2 or SEQ ID NO: 4, sufficient to provide a binding site for an anti-HGF-AIh antibody, that is fused to an heterologous amino acid sequence.

24. The chimeric molecule of claim 23, wherein said heterologous amino acid sequence is an epitope tag sequence.

25. The chimeric molecule of claim 23, wherein said heterologous amino acid sequence is an Fc region of an immunoglobulin.

26. An antibody which specifically binds to an epitope of a polypeptide of SEQ ID NO: 2 or SEQ ID NO: 4.

27. The antibody of claim 26, where said antibody is a monoclonal antibody.

28. The antibody of claim 26, wherein said antibody is a humanized antibody.

29. An agonist to an HGF-AIh.

30. An antagonist to an HGF-AIh.

31. A composition comprising a therapeutically effective amount of an active agent selected from the group consisting of: (a) an HGF-AIh polypeptide, (b) an agonist to an HGF-AIh polypeptide, (c) an antagonist to an HGF-AIh polypeptide, and (d) an anti-HGF-AIh antibody; in combination with a pharmaceutically acceptable carrier.

32. A method of treating a cellular proliferative disorder by administring a therapeutically effective amount of an HGF-AIh polypeptide, agonist, or antagonist thereof to a mammal with said disorder.

33. An article of manufacture comprising a container, label and therapeutically effective amount of HGF-AIh agonist or antagonist thereof in combination with a pharmaceutically effective carrier.
